(19) United States
(12) Patent Application Publication
(10) Pub. No.: US 2024/0118165 A1
(43) Pub. Date: Apr. 11, 2024

(54) DEVICE MEASURING THE DEFOCUS CURVE OF A MULTIFOCAL INTRAOCULAR LENS

(71) Applicant: THE CATHOLIC UNIVERSITY OF KOREA INDUSTRY-ACADEMIC COOPERATION FOUNDATION, Seoul (KR)

(72) Inventor: Hosik Hwang, Seoul (KR)

(73) Assignee: THE CATHOLIC UNIVERSITY OF KOREA INDUSTRY-ACADEMIC COOPERATION FOUNDATION, Seoul (KR)

(21) Appl. No.: 18/377,504

(22) Filed: Oct. 6, 2023

(30) Foreign Application Priority Data
Oct. 6, 2022 (KR) 10-2022-0127517

(51) Int. Cl. G01M 11/02; A61F 2/16

(52) U.S. Cl. CPC G01M 11/0257; A61F 2/1618

(57) ABSTRACT

Disclosed is a defocus curve measuring apparatus for a multifocal intraocular lens capable of automatically measuring an objective defocus curve. A defocus curve measuring apparatus for a multifocal intraocular lens according to an aspect of the present invention may include a light source passed through a target image for focusing; a first lens that infinitely refracts the focus as the light source passes through it; a beam splitter having a polarization function that passes p-polarized light of the light source that has passed through the first lens and reflects s-polarized light; an intraocular lens module that refracts the light source that has passed through the beam splitter as it passes, thereby determining the focus; and a camera that captures an image of s-polarized light reflected by the beam splitter after the light source reflected from a retina passes through the intraocular lens module.

### CROSS-REFERENCE TO RELATED APPLICATION

This application claims priority to and the benefit of Korean Patent Application No. 10-2022-0127517, filed on Oct. 6, 2022, the disclosure of which is incorporated herein by reference in its entirety.

### TECHNICAL FIELD

The present invention relates to a defocus curve measuring apparatus for a multifocal intraocular lens, and more particularly, to a defocus curve measuring apparatus for a multifocal intraocular lens capable of automatically measuring an objective defocus curve.

### BACKGROUND

In general, the use of multifocal intraocular lenses is increasing day by day. After inserting a multifocal intraocular lens, a survey is conducted to evaluate the multifocal intraocular lens, including distance visual acuity, near visual acuity, independence of glasses, satisfaction, and recommendation. Among them, one of the most important data that shows the function of a multifocal intraocular lens is the defocus curve. This shows how one's visual acuity is at far, intermediate, and near distances.

In order to obtain the defocus curve of the multifocal intraocular lens in the eye of a patient implanted with a multifocal intraocular lens, repeated visual acuity measurements must be made using the fogging method. However, this is cumbersome for patients and examiners, and even at the same defocus curve, visual acuity deviation is severe for each patient, making it inaccurate. It would be good if there was equipment that could automatically and objectively measure the defocus curve, such as an autorefractometer that measures the refractive error in a patient's eye.

It would be nice if it could be measured with a type of machine such as an autorefractometer, but this is currently not possible. The first reason is that it cannot obtain a defocus curve and simply measures the refractive error. When a multifocal intraocular lens is inserted, in principle, two refractive errors and multiple focuses should be measured, but it seems that only the refractive error of the far distance is measured. Second, because it uses infrared rays for patient safety, diffractive IOLs (intraocular lenses), which are widely used these days, are designed based on visible light and cannot be measured with this equipment.

Obtaining a defocus curve through an optical bench test is objective, but this method cannot be applied to patients because it analyzes images that have passed through an intraocular lens (IOU. This is because a camera cannot be placed inside the eye behind the intraocular lens (IOU.

### SUMMARY OF THE INVENTION

### Technical Problem

The present invention is to solve the above problems, and the present invention is directed to providing a defocus curve measuring apparatus for a multifocal intraocular lens that can obtain a defocus curve of a multifocal intraocular lens model eye by entering light from the outside of the model eye into which the multifocal intraocular lens is inserted and analyzing it from the outside so that it can be applied directly to patients.

The problems of the present invention are not limited to those mentioned above, and other problems not mentioned will be clearly understood by those of ordinary skill in the art from the following description.

### Technical Solution

According to an aspect of the present invention, a defocus curve measuring apparatus for a multifocal intraocular lens may include a light source passed through a target image for focusing; a first lens that infinitely refracts the focus as the light source passes through it, a beam splitter having a polarization function that passes p-polarized light of the light source that has passed through the first lens and reflects s-polarized light; an intraocular lens module that refracts the light source that has passed through the beam splitter as it passes, thereby determining the focus; and a camera that captures an image of s-polarized light reflected by the beam splitter after the light source reflected from a retina passes through the intraocular lens module.

In this case, the light source and the target image may be installed to be movable.

In this case, the first lens may be a badal lens.

In this case, an artificial cornea may be provided between the beam splitter and the intraocular lens module.

In this case, a second lens may be provided between the beam splitter and the camera.

In this case, the distance between the intraocular lens module and the retina may be adjusted.

In this case, a third lens with variable focus may be provided between the intraocular lens module and the beam splitter.

In this case, the third lens may be a liquid lens.

In this case, the intraocular lens module may include a main body having a through hole formed therein; an intraocular lens mounted to be fixed inside the through hole of the main body; a window member fixed at upper and lower ends of the through hole of the main body so that the inside of the through hole of the main body is sealed, and made of a transparent material; a fixing part that fixes the position of the intraocular lens; and an aqueous liquid filled inside the through hole.

In this case, a cross-section of the through hole may be formed in a circular shape, and a central axis of the intraocular lens may be disposed on the central axis of the through hole.

In this case, the fixing part may include an upper fixing adapter and a lower fixing adapter installed on upper and lower parts of the intraocular lens to fix the intraocular lens at a certain height inside the through hole, and an assembling jaw having a reduced diameter may be formed at a lower end of the through hole of the main body, and the lower fixing adapter, the intraocular lens, and the upper fixing adapter may be sequentially stacked over the assembling jaw.

### Advantageous Effects

According to the above configuration, the defocus curve measuring apparatus for a multifocal intraocular lens according to an exemplary embodiment of the present invention can automatically measure an objective defocus curve from outside a patient for a target multifocal intraocular lens.

### BRIEF DESCRIPTION OF THE DRAWINGS

FIG. 1 is a mimetic diagram of a light source passing through to the retina in a defocus curve measuring apparatus for a multifocal intraocular lens according to an exemplary embodiment of the present invention.

FIG. 2 is a mimetic diagram of a light source passing through from the retina to the camera in a defocus curve measuring apparatus for a multifocal intraocular lens according to an exemplary embodiment of the present invention.

FIG. 3 is a cross-sectional view of an intraocular lens module, which is a component of a defocus curve measuring apparatus for a multifocal intraocular lens according to an exemplary embodiment of the present invention.

FIG. 4 is a configuration diagram showing a defocus curve measuring apparatus for a multifocal intraocular lens according to another exemplary embodiment of the present invention.

### DETAILED DESCRIPTION OF THE EMBODIMENTS

Hereinafter, exemplary embodiments of the present invention will be described in detail so that those of ordinary skill in the art can readily implement the present invention with reference to the accompanying drawings. The present invention may be embodied in many different forms and is not limited to the embodiments set forth herein. In the drawings, parts unrelated to the description are omitted for clarity of description of the present invention, and throughout the specification, same or similar reference numerals denote same elements.

Terms and words used in the present specification and claims should not be construed as limited to their usual or dictionary definition, and they should be interpreted as a meaning and concept consistent with the technical idea of the present invention based on the principle that inventors may appropriately define the terms and concept in order to describe their own invention in the best way.

Accordingly, the embodiments described in the present specification and the configurations shown in the drawings correspond to preferred embodiments of the present invention, and do not represent all the technical idea of the present invention, so the configurations may have various examples of equivalent and modification that can replace them at the time of filing the present invention.

It should be understood that the terms “comprise” or “have” or the like when used in this specification, are intended to describe the presence of stated features, integers, steps, operations, elements, components and/or a combination thereof but not preclude the possibility of the presence or addition of one or more other features, integers, steps, operations, elements, components, or a combination thereof.

The presence of an element in/on “front”, “rear”, “upper or above or top” or “lower or below or bottom” of another element includes not only being disposed in/on “front”, “rear”, “upper or above or top” or “lower or below or bottom” directly in contact with other elements, but also cases in which another element being disposed in the middle, unless otherwise specified. In addition, unless otherwise specified, that an element is “connected” to another element includes not only direct connection to each other but also indirect connection to each other.

Hereinafter, a defocus curve measuring apparatus for a multifocal intraocular lens according to an exemplary embodiment of the present invention will be described in detail with reference to the drawings.

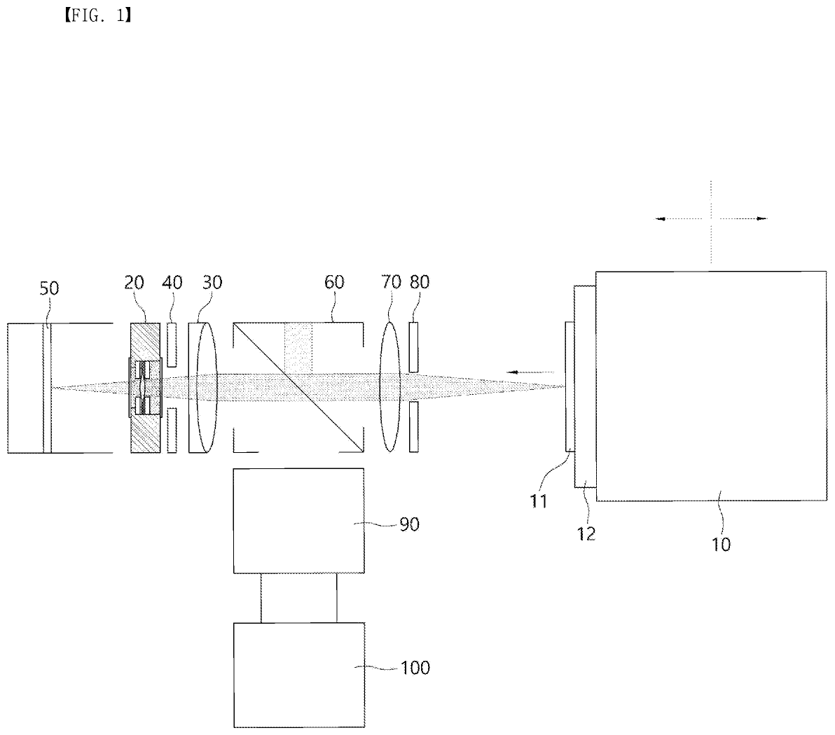

Referring to FIGS. 1 to 3, the defocus curve measuring apparatus for a multifocal intraocular lens according to an exemplary embodiment of the present invention may include a light source 12, a first lens 70, a beam splitter 60, an intraocular lens module 20, and a camera 100.

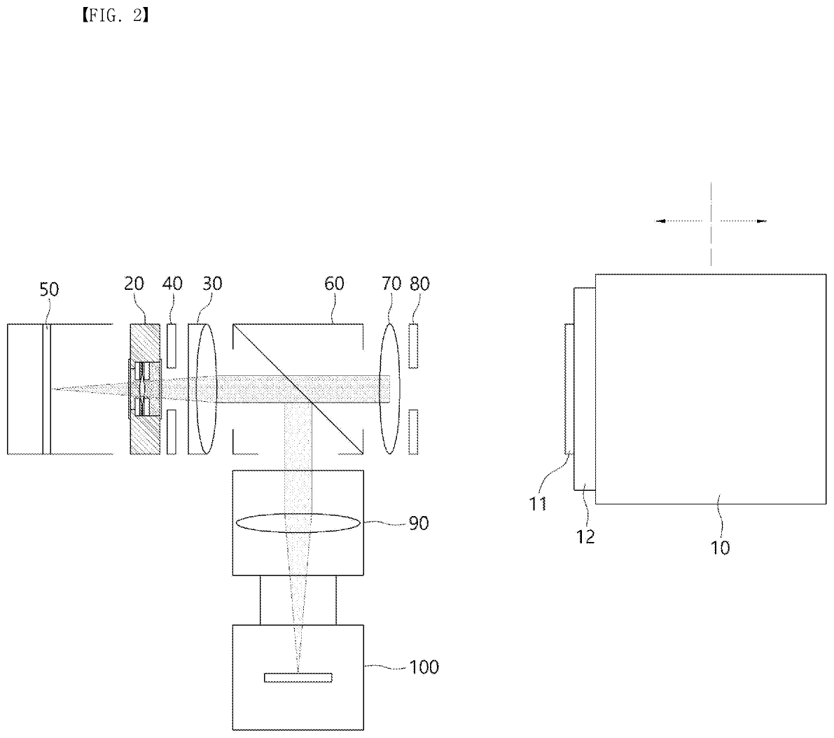

Referring to FIGS. 1 to 2, the light source 12 passes through a target image 11 for focusing to measure the defocus curve.

In this case, an LED may be applied as a light source, and USAF 1951 is provided as the target image 11 on the front of the LED. Therefore, the LED light source 12 passes through the target image 11 and proceeds. Of course, at this time, it is not polarized.

In this case, the light source 12 and the target image 11 may be installed to be movable. The defocus error to obtain the defocus curve may be created by continuously adjusting the distance. This movement of the light source 12 and the target image 11 can be achieved by building equipment that can move the stage 10 on which the LED and the target image are fixed, back and forth in a straight direction. Such mechanical equipment is generally possible through mechanical design using the driving force of a step motor.

In this case, the LED light source 12 that passes through the target image 11 passes through a front aperture 80.

Referring to FIGS. 1 and 2, the first lens 70 refracts the focus to infinity as the light source 12 passes through it.

In this case, the first lens 70 may be a badal lens. Due to its nature, the badal lens can focus the input light source infinitely and fire it.

Referring to FIGS. 1 and 2, the beam splitter 60 has a polarization function that passes the p-polarized light of the light source 12 that has passed through the first lens 70 and reflects the s-polarized light.

In this case, a linear polarizer may be added to the beam splitter 60. This is because the beam splitter 60 cannot completely divide p-polarized light and s-polarized light.

In this case, an artificial cornea 30 may be provided between the beam splitter 60 and the intraocular lens module 20. In addition, the aperture 40 may be reinstalled between the intraocular lens module 20 and the artificial cornea 30.

Referring to FIGS. 1 to 3, the intraocular lens module 20 may refract the light source that has passed through the beam splitter 60 as it passes, thereby determining the focus.

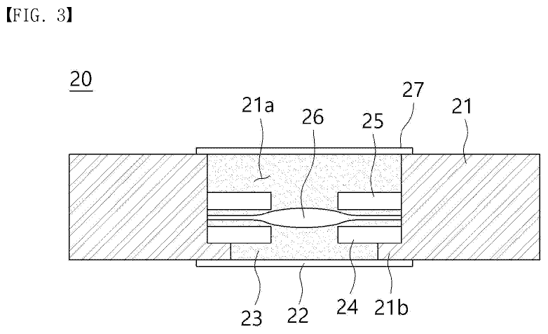

In this case, referring to FIG. 3, the intraocular lens module 20 may include a main body 21, an intraocular lens 26, a window member 22 and 27, a fixing part, and an aqueous liquid 23.

Referring to FIG. 3, the main body 21 has a through hole 21a formed in the center in the up and down direction.

In this case, the cross-section of the through hole 21a may be formed in a circular shape.

In this case, the main body 21 may have a substantially flat cylindrical shape. However, the shape of the main body is not limited to a cylindrical shape, and may have a rectangular parallelepiped shape or a polyhedral shape depending on the shape.

In this case, an assembling jaw 21b having a reduced diameter is formed at the lower end of the through hole 21a of the main body 21, and the lower fixing adapter 24, the intraocular lens 26, and the upper fixing adapter 25 are sequentially stacked over the assembling jaw 21b.

Referring to FIG. 3, the intraocular lens 26 is mounted horizontally so as to be fixed inside the through hole 21a of the main body 21. Of course, the intraocular lens 26 is stably fixed and maintained in that state by the upper and lower fixing adapters 24 and 25.

Referring to FIG. 3, the window member 22 and 27 is fixed at upper and lower ends so that the inside of the through hole 21a of the main body 21 is sealed, and made of a transparent material.

In this case, the window member 22 and 27 may be manufactured using glass or synthetic resin panels. In addition, the window member 22 and 27 may be fixed to the main body 21 by being attached to the main body 21 by an adhesive.

Referring to FIG. 3, the fixing part fixes the position of the intraocular lens 26.

In this case, the fixing part is composed of an upper fixing adapter 25 and a lower fixing adapter 24, and the intraocular lens 26 is positioned therebetween to maintain a fixed state. That is, the intraocular lens 26 is positioned between the upper and lower fixing adapters 24 and 25, but is indirectly fixed such that the intraocular lens 26 itself is not fixed to the main body 21 but the upper and lower fixing adapters 24 and 25 are fixed.

In this case, the upper and lower fixing adapters 24 and 25 may be in the form of a flat annular ring like a washer, and a method in which the upper fixing adapter 25 is fixed by tightening the headless bolt from the side, or a method of fixing an annular fixing bolt that is screwed onto the upper fixing adapter 25 may be applied. In addition to these fixing methods, by fixing the upper fixing adapter 25 in various ways, it is possible to stably fix the intraocular lens 26 at a predetermined position inside the main body 21.

The aqueous liquid 23 may be water or physiological saline solution filled inside the through hole 21a.

Referring to FIGS. 1 and 2, the target image 11 of the light source that has passed through the intraocular lens module 20 is formed on the retina 50. Of course, the formed target image 11 will reflect the target image, with its polarization disappearing as it is reflected.

In this case, the distance between the intraocular lens module 20 and the retina 50 may be adjusted. This helps to create an appropriate defocus error.

Referring to FIGS. 1 and 2, the camera 100 may capture an image of s-polarized light reflected by the beam splitter 60 after the light source reflected from the retina 50 passes through the intraocular lens module 20.

In this case, a second lens 90 may be provided between the beam splitter 60 and the camera 100. The second lens 90 may use various lenses or combinations of lenses so that the camera 100 can achieve good focus.

Referring to FIG. 1, a mimetic diagram of a light source passing through to the retina 50 in a defocus curve measuring apparatus for a multifocal intraocular lens according to an exemplary embodiment of the present invention is shown. The distance is adjusted by the stage 10, so that the target image 11, which is a visual target, due to the fixed LED light source 12 and the target image 11, passes through the aperture 80 and the badal lens 70 and then partially passes through the beam splitter 60. In this case, s-polarized light is reflected, and p-polarized light passes through and proceeds. Then, after passing through the artificial cornea 30, the aperture 40, and the intraocular lens module (IOL) 20 of the model eye, the p-polarized light forms an image on the retina 50. This is the first pass.

Referring to FIG. 2, a mimetic diagram of a light source passing through from the retina 50 to the camera 100 in a defocus curve measuring apparatus for a multifocal intraocular lens according to an exemplary embodiment of the present invention is shown. The image formed on the retina 50 is reflected in a state in which polarization is lost, and then again passes through the model eye's intraocular lens (IOL) 20, aperture 40, and artificial cornea 30, and then at the beam splitter 60, the p-polarized light passes through the beam splitter 60 and goes straight, and the s-polarized light is reflected and passes through the second lens 90, which is an imaging lens to form an image on the sensor of the CMOS camera 100. This is the second pass. This is a kind of double pass system.

Referring to FIG. 3, it shows a cross-sectional view of an intraocular lens module, which is a component of a defocus curve measuring apparatus for a multifocal intraocular lens according to an exemplary embodiment of the present invention. In the present invention, the beam splitter used was a polarizing plate beam splitter (25 mm Square Broadband Polarizing Plate Beamsplitter, Edund optics). This is to eliminate excessive reflection occurring in the two windows 22 and 27 of the wet cell of the intraocular lens. Due to this excessive reflection, the image formed on the sensor of the camera 100 (USAF resolution target image) appears blurry. When light enters the beam splitter 60, p-polarized light (p-pol) proceeds and s-polarized light (s-pol) is reflected. The p-polarized light that proceeded is reflected from the two windows 22 and 27 of the model eye, and after reflection, the p-polarized light is maintained and passes through the beam splitter 60 and proceeds. The light source that reaches the retina 50 loses polarization as it scatters on the paper, and when it passes through the beam splitter 60 again, the p-polarized light passes through and the s-polarized light is reflected and enters the camera 100 through the second lens 90. Through this, blurring of the image due to unwanted excessive reflection occurring in the windows 22 and 27 of the model eye can be minimized. Since the beam splitter 60 does not split 100% into p-polarized light and s-polarized light, a linear polarizer can be added. It may be positioned between the target image (USAF) and the beam splitter 60 so that all p-polarized light passes through, and in front of the camera 100, it may be positioned so that only s-polarized light passes through.

As a result, it was possible to obtain a defocus curve that was very similar to the defocus curve obtained by shooting without a screen.

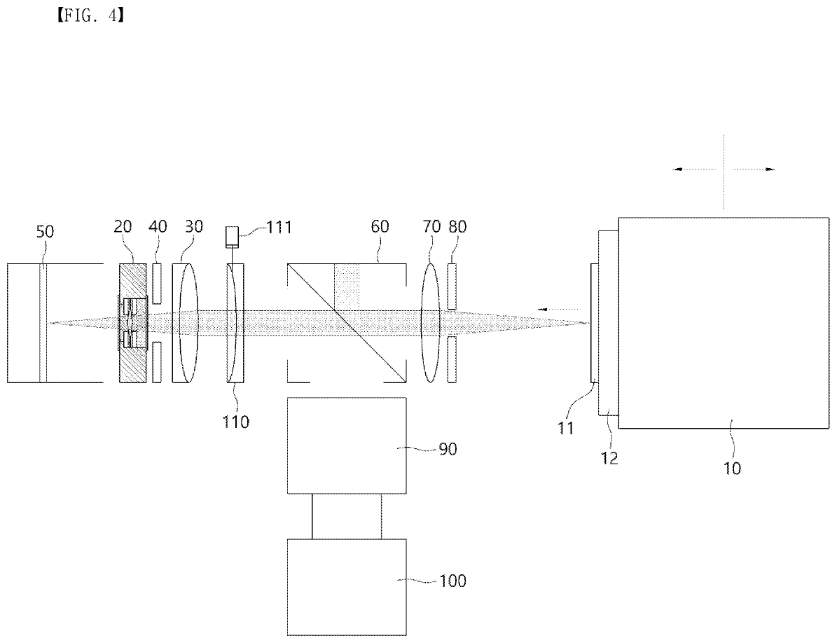

Meanwhile, FIG. 4 is a configuration diagram showing a defocus curve measuring apparatus for a multifocal intraocular lens according to another exemplary embodiment of the present invention. Here, a third lens 110 with variable focus may be provided between the intraocular lens module 20 and the beam splitter 60.

In this case, the third lens 110 may be a liquid lens. A lens whose focus can vary depending on the amount of liquid filled inside by an actuator 111 may be applied. Of course, the application of such a liquid lens can replace the repetitive work of moving and returning components to separately adjust the focal length.

Although exemplary embodiments of the present invention have been described, the idea of the present invention is not limited to the embodiments set forth herein. Those of ordinary skill in the art who understand the idea of the present invention may easily propose other embodiments through supplement, change, removal, addition, etc. of elements within the same idea, but the embodiments will be also within the idea scope of the present invention.

<Description of Symbols>

| | |
|---|---|
| 10: stage | 11: target image |
| 2: light source | 20: intraocular lens module |
| 30: artificial cornea | 40, 80: aperture |
| 50: retina | 60: beam splitter |
| 70: first lens | 90: second lens |
| 100: camera | |

What is claimed is:

1. A defocus curve measuring apparatus for a multifocal intraocular lens, comprising:
a light source passed through a target image for focusing;
a first lens that infinitely refracts the focus as the light source passes through it;
a beam splitter having a polarization function that passes p-polarized light of the light source that has passed through the first lens and reflects s-polarized light;
an intraocular lens module that refracts the light source that has passed through the beam splitter as it passes, thereby determining the focus; and
a camera that captures an image of s-polarized light reflected by the beam splitter after the light source reflected from a retina passes through the intraocular lens module.

2. The defocus curve measuring apparatus for a multifocal intraocular lens of claim 1, wherein the light source and the target image is installed to be movable.

3. The defocus curve measuring apparatus for a multifocal intraocular lens of claim 1, wherein the first lens is a badal lens.

4. The defocus curve measuring apparatus for a multifocal intraocular lens of claim 1, wherein an artificial cornea is provided between the beam splitter and the intraocular lens module.

5. The defocus curve measuring apparatus for a multifocal intraocular lens of claim 1, wherein a second lens is provided between the beam splitter and the camera.

6. The defocus curve measuring apparatus for a multifocal intraocular lens of claim 1, wherein the distance between the intraocular lens module and the retina can be adjusted.

7. The defocus curve measuring apparatus for a multifocal intraocular lens of claim 1, wherein a third lens with variable focus is provided between the intraocular lens module and the beam splitter.

8. The defocus curve measuring apparatus for a multifocal intraocular lens of claim 7, wherein the third lens is a liquid lens.

9. The defocus curve measuring apparatus for a multifocal intraocular lens of claim 1, wherein the intraocular lens module comprises:
a main body having a through hole formed therein;
an intraocular lens mounted to be fixed inside the through hole of the main body;
a window member fixed at upper and lower ends of the through hole of the main body so that the inside of the through hole of the main body is sealed, and made of a transparent material;
a fixing part that fixes the position of the intraocular lens; and
an aqueous liquid filled inside the through hole.

10. The defocus curve measuring apparatus for a multifocal intraocular lens of claim 9, wherein a cross-section of the through hole is formed in a circular shape, and a central axis of the intraocular lens is disposed on the central axis of the through hole.

11. The defocus curve measuring apparatus for a multifocal intraocular lens of claim 9,
wherein the fixing part comprises an upper fixing adapter and a lower fixing adapter installed on upper and lower parts of the intraocular lens to fix the intraocular lens at a certain height inside the through hole, and
an assembling jaw having a reduced diameter is formed at a lower end of the through hole of the main body, and the lower fixing adapter, the intraocular lens, and the upper fixing adapter are sequentially stacked over the assembling jaw.
